(19) United States
(12) Patent Application Publication
(10) Pub. No.: US 2011/0111388 A1
(43) Pub. Date: May 12, 2011

(54) METHOD FOR THE SENSITIVE DETECTION OF POLYAMINO ACIDS AND OTHER MACRO-MOLECULES

(76) Inventors: Horst Hennig, Leipzig (DE); Ralf Hoffmann, Grossposna (DE); Jörg Marx, Mohlau (DE); Frank Schumer, Leipzig (DE); Johannes Zeiser, Leipzig (DE); Thole Züchner, Leipzig (DE)

(21) Appl. No.: 12/735,603

(22) PCT Filed: Jan. 24, 2009
(86) PCT No.: PCT/EP2009/000447

§ 371 Date: Dec. 6, 2010

(30) Foreign Application Priority Data
Jan. 29, 2008 (DE) 10 2008 006 610.9

(51) Int. Cl. C12Q 1/70; C12Q 1/68; G01N 33/53; G01N 33/566; G01N 33/00; G01N 21/76; G01N 27/447

(52) U.S. Cl. 435/5; 435/6; 435/7.92; 436/501; 436/86; 436/172; 204/450; 204/461

(57) ABSTRACT

The invention relates to a method for the detection of an analyte containing polyamino acid. The object of the invention is to detect polyamino acids in a highly sensitive manner. The object is achieved in that an LM is coupled to a protein after chemical activation, and is complexed using a lanthanoid ion. In order to detect polyamino acids, the time resolved fluorescence measurement is utilized after electrophoretic separation. The detection limit is 0.5 pg per spot (bovine serum albumin), wherein the linear region extends across six orders of magnitude. The invention also relates to analytes containing nucleic acid.

The invention relates to a method for detecting an analyte, which preferably contains polyamino acids or other macromolecules, by luminescence marking in gels and on solid carriers with use of lanthanoide ions, such as for example europium (III)-, terbium (III)-, samarium (III)-, neodymium (III)-, or dysprosium (III)-complexes.

The detection and the analysis of polyamino acids are important with different commercial and scientific applications. In the following, each homopolymer or heteropolymer of amino acids, including peptides, proteins, and nucleic acids are considered as polyamino acid.

Polyamino acids are typically detected and characterized by gel electrophoresis, solution quantification essays or by detection on solid carriers as for example filtration membranes. An example for filtration membranes are nitro cellulose membranes or membranes out of polyvinylidene difluoride (PVDF). Small amounts of polyamino acids are in general not visible to the open eye and have to be marked before they can be localized and identified.

Two of the most usual methods of marking of polyamino acids in gels are the Coomassie-brilliant blue coloration (in the following designated as CBB-coloration) and silver coloration. The silver coloration is about 100 to 1000 times more sensitive than the CBB-coloration with certain polyamino acids, both colorations however have in common several disadvantages. The CBB-coloration and the silver coloration are both relatively insensitive colorations and have only a narrow region of a linear quantification for the densitometric evaluation. In addition, the marked gels cannot be blotted for further running analyses.

In addition, both the CBB-coloration and the silver coloration require colorimetric detection methods, that is the proteins are detected by the presence of marked or, respectively, opaque bands in the electrophoresis gel. The employment of luminescent reagents for detecting proteins offers the possibility of a strongly increased sensitivity and of a larger linear quantification region, while at the same time the simplicity of the application of the marking reagent is increased. “Luminescent” refers to each reagent which shows luminescence, that is phosphorescence, fluorescence, chemo luminescence or electroluminescence.

Fluorescent reagents have already been employed for marking of polyamino acids, such as for example the dye nile red (9-diethylamino-5H-benzo(alpha)phenoxazine-5-one) (compare Daban et al., ANAL. BIOCHEM. 199, 169 (1991)). Further examples of frequently employed fluorescent reagents belong to the family of the cyanine dyes (also called Cy-dyes) (compare Ernst L A, Gupta R K, Mujumdar R B, Waggoner A S. Cyanine dye labeling reagents for sulthydryl groups. Cytometry. 1989; 10(1):3-10). Cyanine dyes have been employed amongst others also as fluorescent dyes for polyamino acids in gels, on membranes and other carriers. While the marking with the low molecular organic dyes runs very quickly and is relatively insensitive with respect to the composition of the polyamino acids and requires no decoloration or bleaching, organic fluorescence dyes typically suffer from the disadvantage of a high background coloration on solid carriers and gels and of a quick bleaching upon illumination.

All known marking methods for polyamino acids in gels and on solid carriers however have a relatively poor detection limit in the lower nanogram up to the upper piko gram region (ng to pg). This holds also for the most sensitive of the presently known marking methods for polyamino acids, such as for example the silver marking, the use of cyanine dyes and others (compare overview: Hirsch J, Hansen K C, Burlingame A L, Matthay M A. (2004) Proteomics: current techniques and potential applications to lung disease. Am J Physiol Lung Cell Mol Physiol. July; 287(1):L1-23 AND Gade D, Thiermann J, Markowsky D, Rabus R (2003). Evaluation of two-dimensional difference gel electrophoresis for protein profiling. J Mol Microbiol Biotechnol; 5(4):240-51.)

The linear regions of the above described dyes extend over three to four orders of magnitude, as described in the above recited publications. At the same time the polyamino acid concentrations in a biological sample may extend over up to 10 orders of magnitude (Sellers T A and Yates J R. Review of proteomics with applications to genetic epidemiology. Genet Epidemiol 24: 83-98, 2003.). Therefore, it is understandable that a marking method is desired which exhibits a linear region of more than four orders of magnitude.

For example, 2D-gels, native and non-native 1D-gels, isoelectrical focusing, dot-blots, slot-blots, differential gel electrophoresis, chromatographic separation techniques, capillary electrophoresis and others belong to the embodiments of usual polyamino acid detection methods. The difficulties of these techniques are situated in the detection of small protein amounts, caused by limitations in the dynamic region of the detection method, and in the identification of an individual polyamino acid in a complex mixture. Biomedical samples, which typically are analyzed, comprise for example body liquids such as plasma serum, spinocortical liquid, blood and others as well as tissue samples of the most different kind. These kinds of samples are complex mixtures of polyamino acids with polyamino acid concentrations in a dynamic region of up to 10 orders of magnitude (Sellers T A and Yates J R. Review of proteomics with applications to genetic epidemiology. Genet Epidemiol 24: 83-98, 2003). The expression and modification changes in the case of the less frequent polyamino acids (“proteins with a small copy number”, 10-1,000 copies for each cell) are possibly the most interesting. Their visualization is frequently covered by strongly expressed polyamino acids (“dominant proteins”, 10,000 and more copies for each cell) (Blackstock W P and Weir M P. Proteomics: quantitative and physical mapping of cellular proteins. Trends Biotechnol 17: 121-127, 1999).

The invention has the object to overcome the disadvantages of the above recited analytical method. The sensitivity of the detection method is to be increased substantially.

It is a purpose of the invention to furnish a method to detect specifically individual polyamino acids or groups of polyamino acids as well as nucleic acids. It is to be tested how far a luminescence marker, which contains complex bound lanthanoids, can be coupled to other polyamino acids, which recognize specifically the polyamino acids to be detected (for example antibodies), and how far a luminescence marker can be coupled directly to the polyamino acids to be detected.

A marking method shall further be presented, which exhibits a linear region of more than four orders of magnitude as well as a high stability against light. The object is resolved according to the present invention corresponding to the main claim.

The use of individual selected lanthanoid complexes as luminescence marker (in the following called LM) is decisive.

The high emission intensity as well as the long lifetime of the electronic excitation states of these compounds opens up the possibility to employ these compounds in the time resolved fluorometry.

The LM comprise a light catching unit (antenna), a frame forming a chelate, a functionality for coupling to polyamine acids as well as a lanthanoid central ion (in the following designated as ln(III)). It is possible to compose the most different combinations of these sub units for the synthesis of LM as described in WO 2005/108405. They offer based on their chemical constitution the basis for an optimization of absorption spectroscopic properties by varying the antenna and the emission by varying of the lanthanoid ion.

The general formula can be presented as follows:

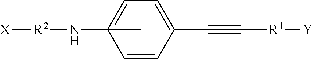

with
X functionality for coupling to a bio molecule
Y functionality for coupling to a bio molecule

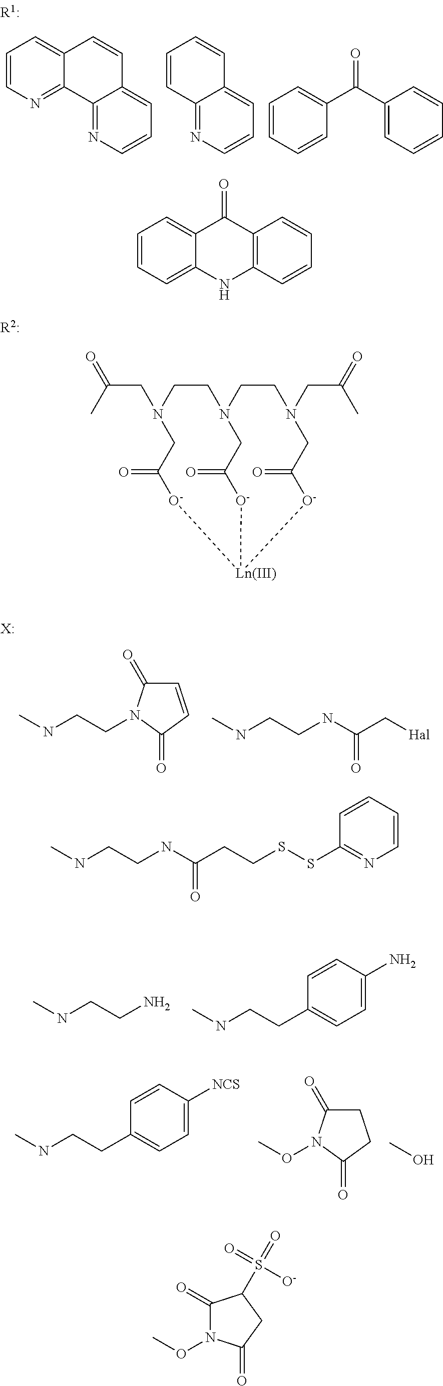

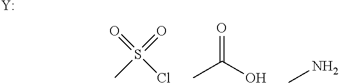

The compounds described in the following are selected from an unusual large number of possible compounds and they have been recognized as suitable for application in the presently claimed analytical method.

After a long search and many experiments, different heterocycles were found of the group of the 2-(4′-amino phenyl ethynyl)-1,10-phenanthroline, differently substituted as a suitable antenna for LM for detection of polyamino acids. More than the one suitable antenna has been recognized from this group, however (6,9-dicarboxy methyl-3-2{(4[1,10]-phenanthroline-2-ylethynylphenyl-carbamoyl)-methyl}-3,6,9-triaza)-undeca-1,11-dicarboxylic acid, in particular for the case where Eu (III) was elected as a lanthanoid.

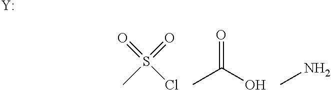

The compounds described in the following are selected from an unusual large number of possible compounds and they have been recognized as suitable for application in the presently claimed analytical method.

After a long search and many experiments, different heterocycles were found of the group of the 2-(4′-amino phenyl ethynyl)-1,10-phenanthroline, differently substituted as a suitable antenna for LM for detection of polyamino acids. More than the one suitable antenna has been recognized from this group, however (6,9-dicarboxy methyl-3-2{(4[1,10]-phenanthrol-2-ylethynylphenyl-carbamoyl)-methyl}-3,6,9-triaza}-undeca-1,11-dicarboxylic acid, in particular for the case where Eu (III) was elected as a lanthanoid.

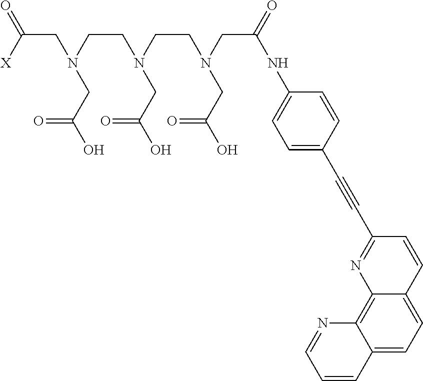

This compound is preferably employed and is designated as LM-prexcursor 1.

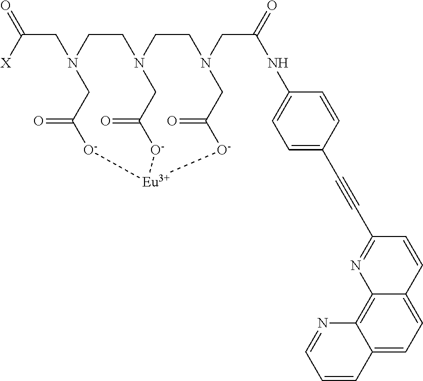

This compound is preferably employed and is designated as LM. 1

The kind of the target polyamino acid is controlled by the coupling functionality. However, maleimide is mainly employed as a covalent sulfhydryl-coupling reagent in gel electrophoresis, since the charge of the marked polyamino acid does not change in this case.

The measurement of the marked polyamino acids while employing time resolved luminescence spectroscopic methods is essential for the present invention.

Time resolved luminescence measurements enable the differentiation between fluorescence and phosphorescence effects. The phosphorescence represents a specific kind of luminescence, which distinguishes from the fluorescence by being of a longer lifetime. A phosphorescent material after light absorption emits longer wave radiation with a time delay. Fluorescent materials after light excitation image in a ns region, whereas phosphorescent materials emit the absorbed radiation in macro to milliseconds. It is therefore possible to measure the phosphorescence separately, even where the measured material or, respectively, the measured sample exhibits a strong fluorescence. This is performed by time delayed measurement after light excitation of the material to be measured or, respectively, of the sample to be measured.

The invention concerns the phosphorescence emission of polyamino acids in gels and on solid carriers with the aid of LM, which contain europium (III), terbium (III), samarium (III), neodymium (III) or dysprosium (III) as a central ion.

A novel polyamino acid detection technology is an essential aspect of the present invention, which technology distinguishes insofar from the conventional marking technique in the handling in that LM is covalently bound to the analyte. Here, the LM is connected covalently to the polyamino acids according to a preferred embodiment of the invention. Another aspect of the invention is the employment of europium (III)-, terbium (III)-, samarium (III)-, neodymium (III)- or dysprosium (III)-central ions for marking of polyamino acids by time delayed detection with significantly higher sensitivity as usual, presently known dyeing methods for polyamino acids with a simultaneously larger linear signal relative to the concentration situation and with higher light stability.

The detection of polyamino acids requires a possibility to detect individual polyamino acids or groups of polyamino acids specifically. This object can be achieved by having the LM directly coupled to other polyamino acids, which recognize specifically the polyamino acid to be detected (for example antibodies), or by having the LM coupled directly to the polyamino acids to be detected. Therefore the coupling of the LM to the polyamino acids is performed covalently in order to assure a high stability of the LM-analyte bond under conditions wherein changes of the following parameters can be imposed: voltage, temperature, pH value, hydrophobicity, enzyme activities, electro-magnetic radiation, interfering organic and inorganic materials, radioactivity and others.

In the following, a covalent connection means an individual covalent bond or combination of stable chemical bonds, possibly comprising single bond, double bond, triple bond or aromatic carbon-carbon bonds as well as carbon-nitrogen bonds, nitrogen-nitrogen bonds, sulfur-nitrogen bonds, carbon-oxygen bonds, carbon-sulfur bonds, phosphorous-oxygen bonds and phosphorous-nitrogen bonds.

Free amino, carboxylate and sulfhydryl groups are suitable for the covalent connection of polyamino acids. According to a first step, the LM-precursor is bound to the polyamino acids with the aid of a coupling function X or Y.

The marking process is finalized by the addition of lanthanoid salt solution to the mixture. The formation of the corresponding complex can be monitored by luminescence or UV-visual spectroscopy.

The present invention uses the above described LM for marking of an analyte, followed by detection of the bond of the LM to the analyte and possibly its quantification or, respectively, of another analysis. The analyte is typically a biomolecule. The analyte is a polyamino acid according to a preferred embodiment of the invention.

The analyte is marked by adding initially In (III)-free LM (in the following designated as LM-precurser) to a sample mixture of which it is assumed that it contains the analyte, such that an optical luminescence effect is observed after aliquot addition of ln(III)-ions and after light excitation.

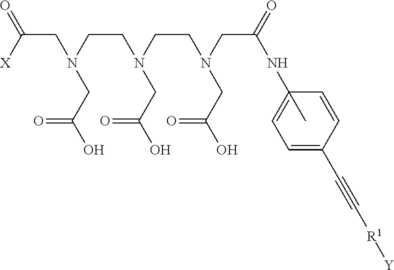

### LM Precursor

According to one aspect of the invention, a quick method for detection of an analyte comprises the following steps:
a) marking of a sample mixture of which it is assumed that it contains the analyte, and which initially is furnished with LM-precursor and addition of an aliquot part of europium (III)-, terbium (III)-, samarium (III), neodymium (III)- or dysprosium (III)-ions in order to form an LM-marked mixture, and wherein each europium (III)-, terbium (III)-, samarium (III)-, neodymium (III)- or dysprosium (III)-coordinated LM independently comprises:
i) a europium (III)-, terbium (III)-, samarium (III)-, neodymium (III)- or dysprosium (III)-ion,
ii) at least one chelate ligand
iii) at least one antenna, and
iv) a coupling function
for the covalent coupling of the LM with a polyamino acid under application of the above recited conditions.
b) incubating the analyte-LM with europium (III)-, terbium (III)-, samarium (III)-, neodymium (III)- or dysprosium (III)-ions in order to assure complete complex formation with selected In (III) ions such that a detectable luminescence occurs after light excitation of the marked analytes.
or a direct use of the luminescence marker covalently coupled to polyamino acids in a polyamino acid analysis process, which shows a detectable luminescence after light excitation;
c) light excitation of the complex of LM/polyamino acid, and
d) the detection of luminescence by way to of a time resolved spectroscopic process.

Additional steps are performed possibly and independent in arbitrary combination, prior to, after, or simultaneously with the marking, in order to take care of a separation or purification of the analyte, in order to increase and reinforce the detection of the analyte, for quantifying the analyte, for identification of a specific analyte or of a group of analytes, for example by employing an immunological reagent such as for example an antibody, an aptamer or a lectin.

The analyte is a biomolecule according to an embodiment of the invention. The analyte is a polyamino acid according to another embodiment. The analyte is a polyamino acid according to another embodiment of the invention, wherein the polyamino acid exhibits post translational modifications. Post translational modifications are defined as chemical modifications of a polyamino acid according to its naturally occurring translation process. Examples for post translational modifications comprise phosphorylization, ubiquitination, methylation, glycosylation, glycation, SUMOylation, acylation, alkylation, methylation, amidation, biotinylation, formylation, carboxylation, glutamylation, glykylation, hydroxylation, isoprenylation, lipoylation, myristoylation, farnesylation, geranylgeranylation, ADP-ribosylation, oxidation, pegylation, phosphopantetheinylation, pyroglutamate formation, sulfation, selenoylation, ISGylation and others.

The analyte is a polyamino acid, which was chemically modified, according to another embodiment, which leads to a polyamino acid modification which does not occur in nature. Examples for chemical modifications which lead to a polyamino acid modification, and which do not occur in nature, are indicated in the ABRF (association of biomolecular resource facilities)-data bank under www.abrf.org.), however these are not limited to the presented examples.

According to another embodiment, the analyte is a biomolecule, which contains at least one nucleic acid.

The analyte is preferably a polymer, and the polymer is a polyamino acid.

The present invention is typically employed in order to detect the desired analyte by combining a sample mixture, which presumably contains the analyte, with a marking material mixture, which in turn is combined with an LM according to the present invention.

It is usually required that one of the recited ln (III)-ions is combined with the LM-precursor in order to obtain a detectable luminescence. The complex formation of the LM-precursor with the ln (III)-ions can occur prior to the coupling at polyamino acids, DNA, RNA, or PNA, however also after the coupling of the LM-precursor to the polyamino acids, DNA, RNA, or PNA with following ln (III)-ion-incubation.

In addition, the incubation of LM with europium (III), terbium (III), samarium (III), neodymium (III) or dysprosium (III), coupled or not coupled, can occur at the following points of time of the analysis:
prior to or after a separation technique is applied to the analysis of a relevant sample;
at a desired arbitrary point in time during the analysis of a sample on a solid or semi-solid matrix. The incubation with the above recited lanthanoid ions can also occur at more than one point in time in order to increase and amplify the signal which is obtained by the optical reaction upon illumination.

After the LM-marking of the analyte, and the sample mixture in the above recited applications is irradiated with a suitable excitation wavelength in order to obtain a detectable luminescence signal. This wavelength is disposed in the region of 280 nm to 400 nm in one situation. This wavelength is disposed in a region of from 350 nm to 370 nm in another situation. According to a preferred aspect of the invention this wavelength is about 360 nm.

After the light excitation of the sample mixture, the luminescence signal is observed at at least one suitable emission wavelength. According to one situation this wavelength is disposed in the region of 280 nm to 800 nm. In another situation this wavelength is disposed in the region of 500 nm 700 nm. These wavelengths of 595 nm and 616 nm are employed according to a preferred aspect of the invention.

The statements are illustrated by way of the drawings.

The solution according to the present invention is realized with a test kit, which is composed as described in the following.

General Description of a Test Kit

Components of the test kit are:
a solution of europium (III)-, terbium (III)-, samarium (III)-, neodymium (III)-, or dysprosium (III)-of a certain concentration,
a solution or a powder of one or several polyamino acids, which are chemically coupled with the LM-precursor as described under point 3.1,
optionally one or several solutions, which are necessary for the dissolving of a polyamino acid in powder form,
a PVDF- or nitrocellulose membrane,
packaging materials and suitable vessels for delivery and application of the kit.

### DESCRIPTION OF THE TEST KIT BY WAY OF EXAMPLES

The test kit contains the following components in a concrete case:
100 microliters of a 10 micro molar europium (III) chloride solution.
one milligram of a lyophilized amount goat-anti-mouse IgG antibody marked with LM-precursor,
100 microliters PBS solution,
a PVDF membrane of the size 30 by 50 cm.

The following steps are necessary for application of the test kit:
a) dissolving the lyophilized antibody in 100 microliters PBS, subjecting to vortexes,
b) Western Blot of a 1D- or 2D-Gel onto the provided PVDF membrane as described in the literature (compare for example http://www.westernblottimg.org/protocols%20western%20blot.htm)
c) blocking of the membrane with for example milk powder, washing steps, incubation of the membrane with primary antibody as desired, washing steps as described in the literature, for example http://www.westernblottimg.org/protocols%20western%20blot.htm)
d) mixing of the lyophilized antibody from step a) with delivered europium (III) chloride solution for each 2 milliliter TBS-T (Tris buffered saline-Tween) the incubation with Western Blot for one hour, three times washing with TBS-T.
e) measurement of the PVDF membrane at 616 nm emission wavelength and 360 nm excitation wavelength.

### Technical and Economic Advantages of the Invention

Selected LM are employed, which are coupled to proteins and complexed with europium ions after chemical activation. The irradiated energy is transferred to the complexed europium ion after excitation in the UV region. The luminescence of the complexed europium ion is measured. Since the phosphorescence of the europium compounds falls clearly slower than the background fluorescence of the membrane, the proteins on the membrane marked with the new LMs are detected very sensitively by time resolved spectroscopic methods. The limits of detection are situated at 0.3 nanograms per band (bovine serum albumin). The limit of detection is situated at 0.5 pg per spot (bovine serum albumin) at direct spot making of the marked proteins on a membrane. The linear region extends over six orders of magnitude. The advantages of the detection method are obvious, in particular in case of the use of membranes. The simultaneous detection of all proteins on the membrane and of individual proteins is therewith also possible by way of antibodies with comparable sensitivity. This could not be accomplished with the up to now known dyeing techniques. Now for example, the proteins recognized by the antibody can be quantified relative to the respective band. Statements relating to foreign proteins in the band of post translational modifications can be quantified therewith. Additional advantages are associated with the wide linear signal relative to the concentration region over six orders of magnitude as well as in the high light stability.

### LEGENDS TO THE FIGURES

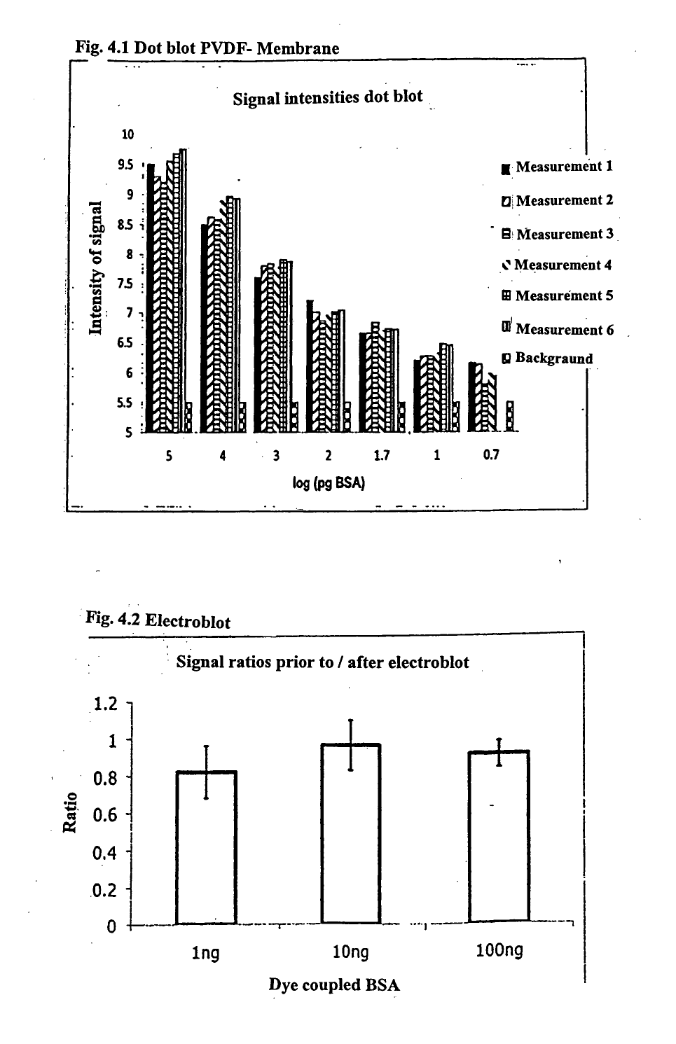

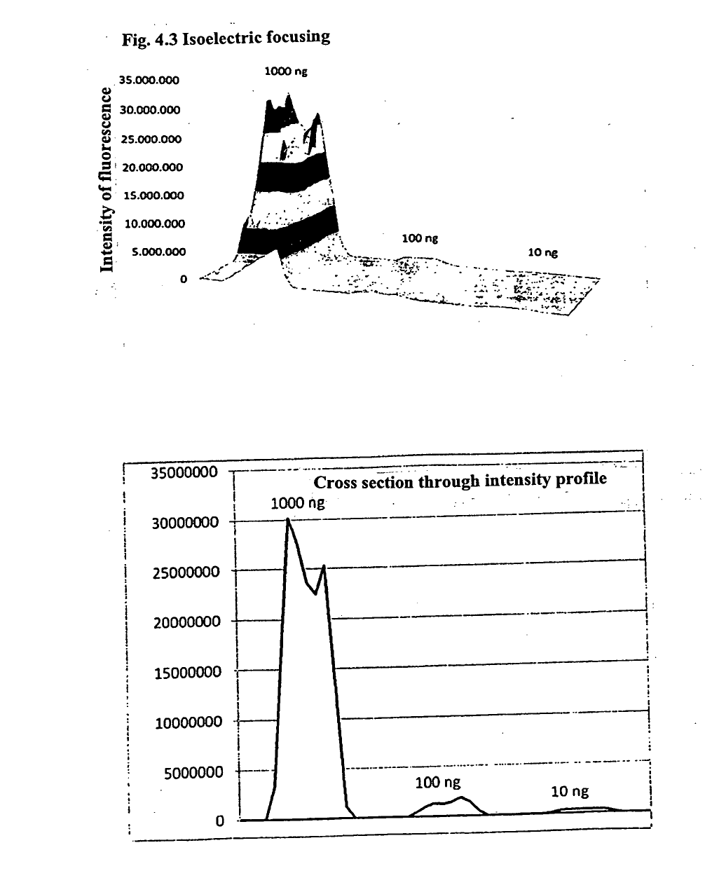

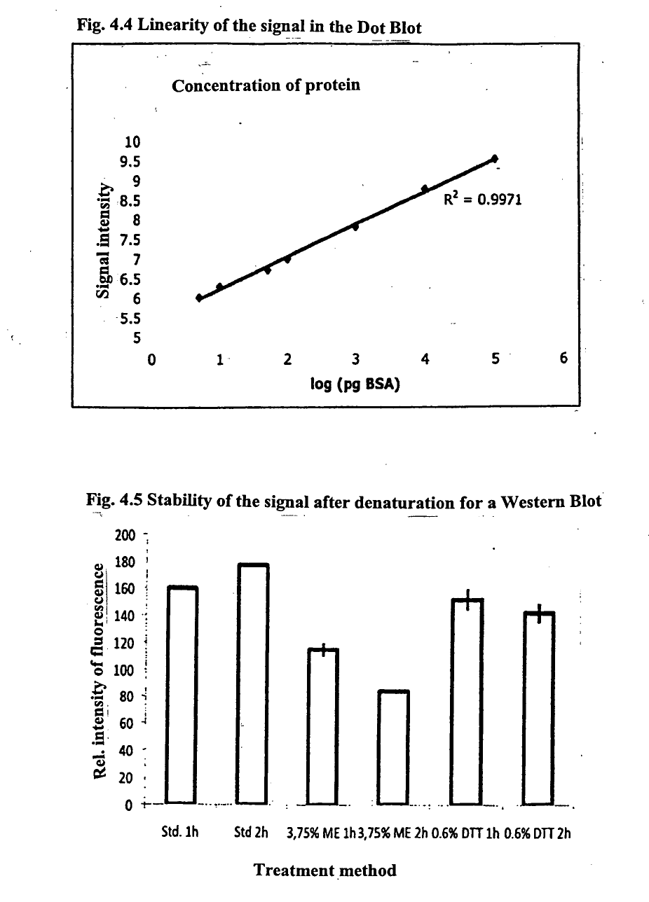

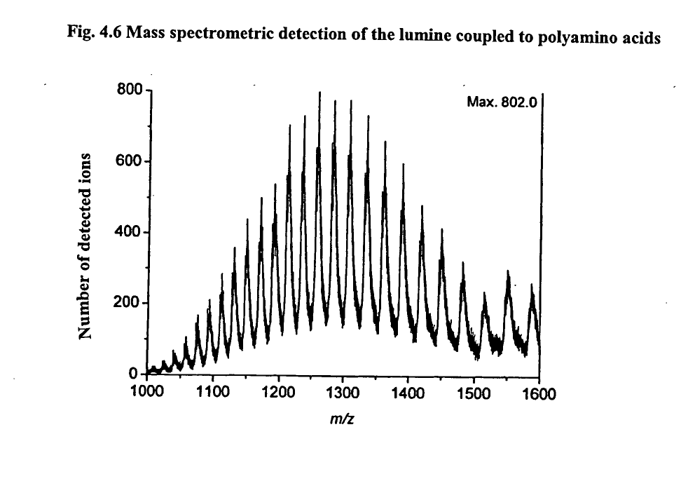

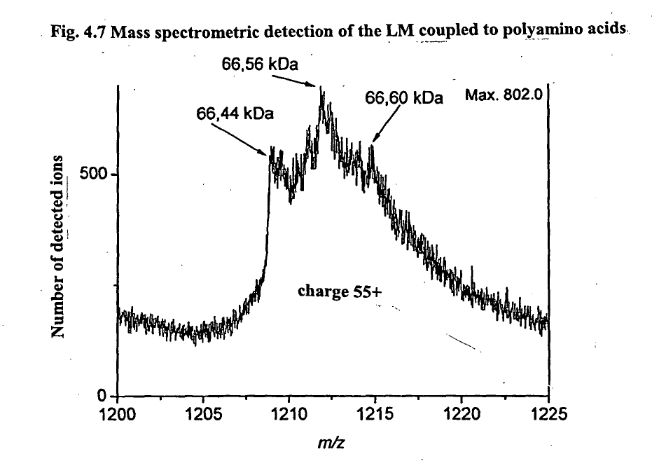

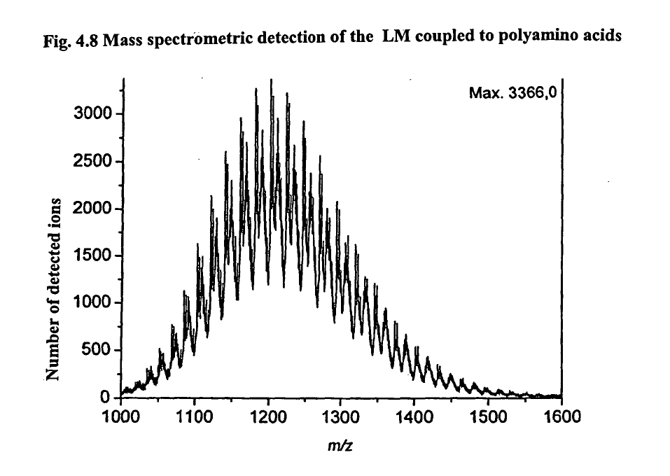

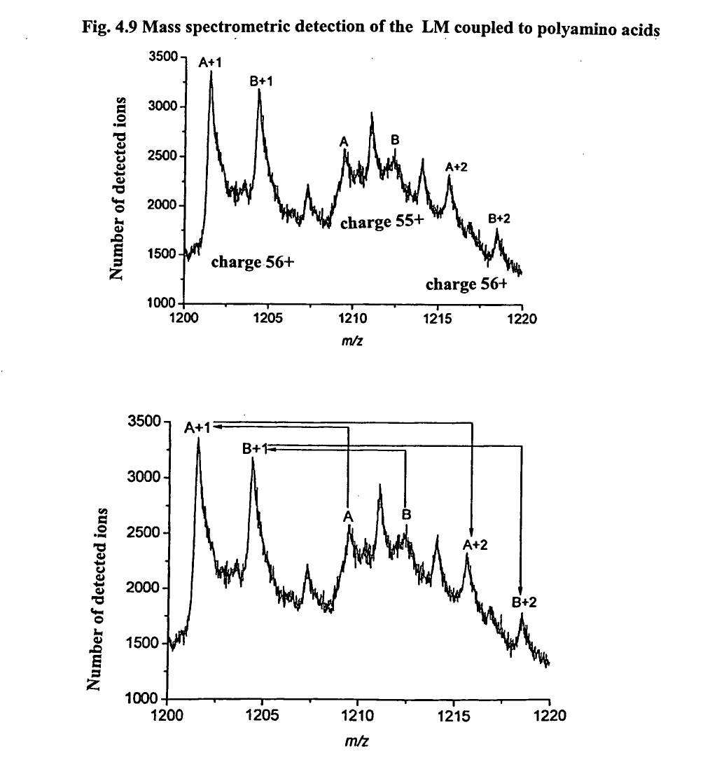

Legend to FIG. 4.1

Signal intensities of BSA (bovine serum albumin) simply marked with LM 1. The samples were spotted and measured on a PVDF membrane, (emission 616 nm, excitation 360 nm). The results of six independent measurements as compared with the background of the membrane are shown. The concentrations of BSA are plotted logarithmic and cover a region of 100,000 up to 5 pg.

Legend to FIG. 4.2

The signal situations are shown prior to and after electro-blot of LM 1 coupled to BSA. For this purpose BSA was marked with LM-precursor and was placed on a PVDF membrane according to the dot blot method, the resulting signals were measured and then this membrane was left in the electro blot method for fifteen minutes at 25V (semi-dry blot). Thereupon, a renewed measurement of the signals was performed. The situations do not significantly distinguish from one, therefore no weakening by electro blot was observed.
BSA=bovine serum albumin

Legend to FIG. 4.3
a) Signal intensity of LM 1 peptide after isoelectric focusing (IEF). Incubation of the LM-precursor 1 with europium (III) before the IEF. The material amounts of the employed peptide walls between 1000 and 10 nanograms. IEF-strip: 7 cm, pH 3-10, linear.

### IEF Conditions:

1) in fifteen minutes to 250 V
2) in 10 hours to 4,000 V (rapid rise)
3) about four hours at 500 V

The following measurement of the strip at 616 nm emission, 360 nm excitation. It is shown that the europium (III) remains complexed by the LM and a signal can be detected depending on the employed concentration.

Legend to FIG. 4.4

Average signal intensities of BSA (bovine serum albumin) once marked with LM 1. The samples were spotted on PVDF membrane and measured. (Emission 616 nm, excitation 360 nm). The averaged results of six independent measurements are shown. The concentrations of BSA are plotted logarithmic and cover a region of 100,000 to 5 pg. This gives a linearity of this signal over six orders of magnitude.

Legend to FIG. 4.5

LM 1-stability after various denaturation methods after one or, respectively, two hours as compared with an untreated sample (standard). RT=room temperature, ME=mercapto ethanol, standard.

Legend to FIG. 4.6

ESI-TOF-mass spectrum of unmarked BSA (bovine serum albumin)

Legend to FIG. 4.7

Section of FIG. 4.6

Legend to FIG. 4.8

ESI-TOF-mass spectrum of BSA (bovine serum albumin) marked with LM-precursor

Legend to FIG. 4.9

Section of FIG. 4.8 mass of the maleimide-activated coupled LM-precursor 1: 793 Da
A: 66468 Da (charge: 55+);
B: 66627 Da (charge: 55+);
A+1: 67231 Da (charge 56+); corresponds to (A)+the LM-mass−31 Da
B+1: 67393 Da (charge 56+); corresponds to (A)+the LM-mass−30 Da
A+2: 68022 Da (charge 56+); corresponds to (A+1)+the LM-mass−1
B+2: 68185 Da (charge 56+); corresponds to (B+1)+the LM-mass+1

1. Method for sensitive detection of polyamino acids and other macromolecules, where a polymer- or other macromolecule is concerned, which contains at least one thiol group or at least one primary or secondary amino group or about a polymer or macromolecule, which can be chemically modified for containing at least one thiol group or at least one primary or secondary amino group, and which comprises the following steps:
a) adding an antenna chelate ligand, designated in the following as LM-precursor, to a sample mixture of which it is assumed that it contains the analyte, which is capable of complexing lanthanoid (III)-ions, in particular europium (III), terbium (III), samarium (III), neodymium (III) or dysprosium (III), which is chemically activated in order to covalently bind the thiol group, the primary amino or the secondary amino groups of the polymer or of the macromolecule,
wherein the antenna chelate ligand is a derivative polyamino polycarbonic acid,
and with an activator A, which is a chemical group which reacts with thiol groups or alternatively reacts with an activator B which represents a chemical group which reacts with primary or secondary amino groups,
b) incubating of the combined mixture for a time period which is sufficient for the prefluorophor to react with the thiol groups, or alternatively with the prefluorophor activated with the activator B in order to react with the primary amino or secondary amino acid in the dye mixture in order to form a derivative of the macromolecule or of the polymer;
c) incubating the combined mixture marked with the LM-precursor for a sufficient time with lanthanoid-(III)-ions, in particular with europium (III)-, terbium (III)-, samarium (III)-, neodymium (III) -or dysprosium (III)-ions for forming a stable complex with the analyte, which carries at least one LM-precursor, complexed with lanthanoid (III)-ions, which furnishes a detectable luminescence emission upon light excitation;
d) separation or partial separation of the analyte mixture in order to allow an analysis of the individual compounds by precipitation, extraction, solid phase extraction, immune dying techniques including immunoassays such as ELISA, dialysis, centrifugation, chromatography, electrophoresis, mass spectrometry, ion mobility, interaction between the analyte and other molecules, in particular macromolecules as for example proteins, DNA, RNA, antibodies, aptameres and phages becomes possible, while the analytes can be directly analyzed or initially be transferred into another solvent, for which is offered electro elution or absorption to a face, which is usually designated as blotting
e) light excitation of the covalent analyte-LM-complex; and
f) detection of the luminescence reaction with time resolved spectroscopic methods.

2. Method according to claim 1, wherein the analyte is a polyamino acid, a peptide, peptidomimetic, a peptide nucleic acid PNA or a protein.

3. Method according to claim 1, wherein the analyte is a polynucleic acid, a single strand DNA, RNA or an oligonucleotide with at least one thiol group and/or primary and/or secondary amino group.

4. Method according to claim 1 which further comprises the quantification of the analyte by time resolved measurement of the detectable luminescence reaction and comparing of the measurement value with a standard.

5. Method according to claim 2, wherein the analyte is disposed on or in a solid or semi solid matrix.

6. Method according to claim 5, wherein the matrix is an electrophoresis gel or a protein binding membrane.

7. Method according to claim 2, which further comprises the electrophoretic separation of the sample mixture prior to combining it, during combining it, or after combining it with the dye mixture.

8. Method according to claim 7, wherein the electrophoretic separation, the irradiation step or the observation step are performed by way of automatic methods.

9. Method according to claim 2 further comprising the analysis of the polyamino acid, of the peptide, of the peptidomimetic, of the peptide nucleic acid PNA or of the protein by way of mass spectrometry.

10. Method according to claim 2 further comprising the analysis of the polyamino acid, of the peptide or of the protein by way of Edman sequencing.

11. Method according to claim 3 further comprising the analysis of the polynucleic acid, of a single strand DNA, of a double strand DNA, RNA, PNA or of an oligonucleotide by mass spectrometry.

12. Method according to claim 3 further comprising the analysis of the polynucleic acid, of a single strand DNA, of a double strand DNA, RNA, PNA, or of an oligonucleotide by sequence analysis.

13. Method according to claim 1 further comprising the addition of an additional reagent to the sample mixture, to the LM-precursor and the corresponding In (III)-ions or the combined mixture.

14. Method according to claim 2 further comprising the detection of a certain polyamino acid, of a certain peptide or proteins or of a class of a polyamino acid, of a peptide, of peptidomimetics, PNA or of a protein with a monoclonal, polyclonal or recombinant antibody or an antibody fragment, an aptamer or phage, which are preferably previously marked according to claim 1.

15. Method according to claim 14 for quantification of the analyte or the analytes comprising polyamino acid, peptide, peptidomimetics, PNA or a protein, relatively to the monoclonal, polyclonal or recombinant antibody, an antibody fragment, an aptamer or a phage, which recognize the analyte.

16. Method according to claim 2, wherein at least two polyamino acid-, peptide- or protein mixtures are marked with different LM, which allows a parallel and independent detection of all analytes in one and the same solution, the same volume or on the same surface in order to quantify the analytes present in each mixture relative to the analytes present in another mixture.

17. Method according to claim 16, wherein the proteins are separately by electrophoresis in order to quantify a certain protein in each of the mixtures which contain another LM.

18. Method according to claim 17, wherein the polyamino acids, the proteins, that peptidomimetics, PNA or the peptides have been transferred onto a membrane.

19. Method according to claim 17, wherein certain proteins are marked additionally with a monoclonal, polyclonal or recombinant antibody, antibody fragment, an aptamer or a phage, which have been previously marked according to claim 1.

20. Method according to claim 18, wherein certain proteins are marked additionally with a monoclonal, polyclonal or recombinant antibody, an antibody fragment, an aptamer or a phage, which have been previously marked according to claim 1.

21. Method according to claim 3, further comprising the detection of a polynucleic acid, a single strand DNA, a double strand DNA, RNA, PNA or of an oligonucleotide with a monoclonal, polyclonal or recombinant antibody, an antibody fragment, an aptamer or a phage or an arbitrary other protein or oligonucleotide, a polynucleic acid, a single strand DNA, a double strand DNA, RNA, or PNA, which can be hybridized and which preferably have previously been marked.

22. Method according to claim 14 for quantifying of the analyte or analytes consisting of RNA or, for recognizing the analyte relative to the employed DNA sensor.

23. Method according to claim 2, wherein at least two DNA- or RNA mixtures have been marked with different LM, which allows for a parallel and independent detection of all analytes in the same solution, in the same volume or on the same surface, in order to quantify the analytes present in each mixture relative to the analytes present in another mixture.

24. Method according to claim 16, wherein the DNA or RNA are separated electrophoretically in order to quantify a certain DNA or RNA in each of the mixtures, which contain a different LM.

25. Method according to claim 17, wherein certain DNA-or RNA are marked additionally with DNA-or RNA-sensor, which had been previously marked.

26. Use of derivatives of the 2-(4′-amino-phenyl-ethynyl)-1,10-phenanthroline as luminescence marker for the determination of biomolecules by time resolved luminescence spectroscopy.

27. Use of derivatives of the 2-(4′-amino-phenyl-ethynyl)-1,10-phenanthroline according to claim 26, characterized in that (6,9-dicarboxy-methyl-3-2{(4[1,10]-phenanthroline-2-ylethynylphenyl-carbamoyl)-methyl}-3,6,9-triaza)-undeca-1,11-dicarboxylic acid, designated as LM-precursor 1, is employed.

28. Test kit for performing the method of claim 1 characterized in that the test kit contains the following components
a) an aqueous solution of a three valued lanthanoid ion
b) one or several polyamino acids, which are chemically coupled with an LM of the group of the 2-(4′-amino-phenyl-ethynyl)-1,10-phenanthrolines, are differently substituted as luminescence markers
c) one or several solutions which are required for the dissolving of a polyamino acid
d) a PVDF- or nitrocellulose membrane
e) auxiliary and packaging agents known in principle.

29. Test kit according to claim 28 characterized in that the test kit confections the following components and contains the components in an arbitrary number
100 microliters of a 10 micromole europium (III) chloride solution
1 mg of a lyophilized luminescence marked goat-anti-mouse-IgG-antibody, wherein the (6,9-dicarboxy methyl-3-2{(4[1,10]-phenanthroline-2-ylethynylphenyl-carbamoyl)-methyl}-3,6,9-triaza)-undeca-1,11-dicarboxylic acid is employed as LM precursor
100 microliters PBS solution
a PVDF-membrane having a size of 30 by 50 cm.
